(19) United States
(12) Patent Application Publication
(10) Pub. No.: US 2020/0137213 A1
(43) Pub. Date: Apr. 30, 2020

(54) EVALUATING ENVIRONMENTAL INFORMATION DURING A TRANSACTION

(71) Applicant: CA, Inc., New York, NY (US)

(72) Inventors: Mohammed Mujeeb Kaladgi, Bangalore (IN); Ruqiya Nikhat Kaladgi, Bangalore (IN); Junaid Ahmed Jamell, Bangalore (IN); Yashwant Ramkishan Sawant, Parbhani (IN)

(21) Appl. No.: 16/171,157

(22) Filed: Oct. 25, 2018

(51) Int. Cl. H04M 1/725; H04W 12/06; H04L 29/08; H04L 29/06; A61B 5/16; A61B 5/00

(52) U.S. Cl. CPC H04M 1/72569; H04W 12/06; H04L 67/1097; H04L 63/10; A61B 2560/0247; A61B 5/4845; A61B 5/6801; H04M 2250/12; A61B 5/165

(57) ABSTRACT

A computer system includes a service terminal, a server computer system, and a mobile device and is used to facilitate a transaction between a user and the service terminal. The service terminal receives authentication information from the user and sends the authentication information to the computer server system. Before transaction resolves, the server computer system sends a request to the mobile device to gather environmental information from one or more sensors in the physical environment of the mobile device. In response, the mobile device identifies sensors, communications with one or more sensors, and receives environmental information. The mobile device sends indications of the environmental information, which the server computer system receives and evaluates. Based on the evaluating, the server computer system determines to alter the transaction, and based on the determination to alter the transaction sends a command altering the transaction to the service terminal.

### BACKGROUND

### Technical Field

This disclosure relates generally to the use of mobile devices and service terminals during transactions.

### Description of the Related Art

Users interact with service terminals to engage in various transactions. For example, a service terminal might facilitate a security transaction with the user as part of granting the user access to a secured area or electronic resource, or facilitate a financial transaction with the user. The service terminal communicates with a server computer system to determine whether to resolve the transaction. To ensure that the user is authorized to engage in the transaction, as part of the transaction, users typically provide one or more pieces of authentication information such as a username or a password.

Mobile devices such as cellular telephones are commonly carried by users and can communicate with the server computer system (e.g., over the Internet). The mobile device may also be used to facilitate various transactions.

### SUMMARY

In various embodiments, after a transaction associated with a user has been initiated at a service terminal, the server computer system communicates with the user's mobile device to gather additional information that the server computer system can use to determine whether to alter the transaction (e.g., by canceling the transaction, by modifying the resolution of the transaction). In such embodiments, the mobile device gathers environmental information from one or more sensors in the physical environment of the mobile device. In many instances, a user will have a mobile device in their physical possession (e.g., by being in the user's pocket or bag), gathering information in the physical environment of the mobile device the mobile device thereby gathers information in the physical environment of the user. Such information may include information about the physical environment (e.g., sound information) or information about the user (e.g., personal metrics), and may be gathered from any of a number of sensors in the physical environment of the mobile device including but not limited to sensors that are integrated into the mobile device, worn by the user, are installed near the service terminal, or are temporarily located near the mobile device. Upon receiving the gathered information, in various embodiments the server computer system uses the information to determine whether to alter the transaction. In some embodiments, the server computer system determines that the user is in duress and can alter the transaction accordingly. In other embodiments, the server computer system determines that the user is intoxicated and can alter the transaction accordingly. In some embodiments, the server computer system determines that the transaction may present a security risk and can alter the transaction accordingly. Based on these determinations, the server computer system sends a command to the service terminal altering the transaction in various embodiments.

### BRIEF DESCRIPTION OF THE DRAWINGS

FIG. 1 is a block diagram illustrating an embodiment of a computer system configured to facilitate a transaction.

FIG. 2 is an expanded block diagram of the physical environment 126 of mobile device 120 of FIG. 1 in accordance with various embodiments.

FIG. 3 is an expanded block diagram of the server computer system 110 of FIG. 1 in accordance with various embodiments.

FIG. 4 is a flowchart illustrating an embodiment of a transaction resolution process in accordance with the disclosed embodiments.

FIG. 5 is flowchart illustrating an embodiment of a server portion of a transaction resolution method in accordance with the disclosed embodiments.

FIG. 6 is flowchart illustrating an embodiment of a mobile device portion of a transaction resolution method in accordance with the disclosed embodiments.

FIG. 7 is a block diagram of an exemplary computer system, which may implement the various components of FIGS. 1, 2, and 3.

This disclosure includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

Within this disclosure, different entities (which may variously be referred to as “units,” “circuits,” other components, etc.) may be described or claimed as “configured” to perform one or more tasks or operations. This formulation—[entity] configured to [perform one or more tasks]—is used herein to refer to structure (i.e., something physical, such as an electronic circuit). More specifically, this formulation is used to indicate that this structure is arranged to perform the one or more tasks during operation. A structure can be said to be “configured to” perform some task even if the structure is not currently being operated. A “computer system configured to receive” is intended to cover, for example, a computer system has circuitry that performs this function during operation, even if the computer system in question is not currently being used (e.g., a power supply is not connected to it). Thus, an entity described or recited as “configured to” perform some task refers to something physical, such as a device, circuit, memory storing program instructions executable to implement the task, etc. This phrase is not used herein to refer to something intangible. Thus, the “configured to” construct is not used herein to refer to a software entity such as an application programming interface (API).

The term “configured to” is not intended to mean “configurable to.” An unprogrammed FPGA, for example, would not be considered to be “configured to” perform some specific function, although it may be “configurable to” perform that function and may be “configured to” perform the function after programming.

Reciting in the appended claims that a structure is “configured to” perform one or more tasks is expressly intended not to invoke 35 U.S.C. § 112(f) for that claim element. Accordingly, none of the claims in this application as filed are intended to be interpreted as having means-plus-function elements. Should Applicant wish to invoke Section 112(f) during prosecution, it will recite claim elements using the “means for” [performing a function] construct.

As used herein, the terms “first,” “second,” etc. are used as labels for nouns that they precede, and do not imply any type of ordering (e.g., spatial, temporal, logical, etc.) unless specifically stated. For example, references to “first” and “second” remote sensors would not imply an ordering between the two unless otherwise stated.

As used herein, the term “based on” is used to describe one or more factors that affect a determination. This term does not foreclose the possibility that additional factors may affect a determination. That is, a determination may be solely based on specified factors or based on the specified factors as well as other, unspecified factors. Consider the phrase “determine A based on B.” This phrase specifies that B is a factor is used to determine A or that affects the determination of A. This phrase does not foreclose that the determination of A may also be based on some other factor, such as C. This phrase is also intended to cover an embodiment in which A is determined based solely on B. As used herein, the phrase “based on” is thus synonymous with the phrase “based at least in part on.”

As used herein, the word “module” refers to structure that stores or executes a set of operations. A module refers to hardware that implements the set of operations, or a memory storing the set of instructions such that, when executed by one or more processors of a computer system, cause the computer system to perform the set of operations. A module may thus include an application-specific integrated circuit implementing the instructions, a memory storing the instructions and one or more processors executing said instructions, or a combination of both.

### DETAILED DESCRIPTION

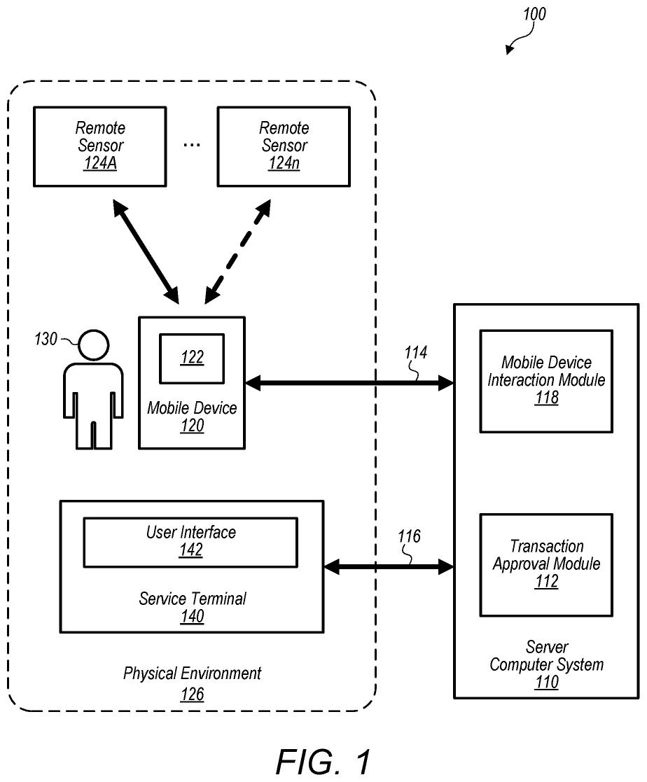

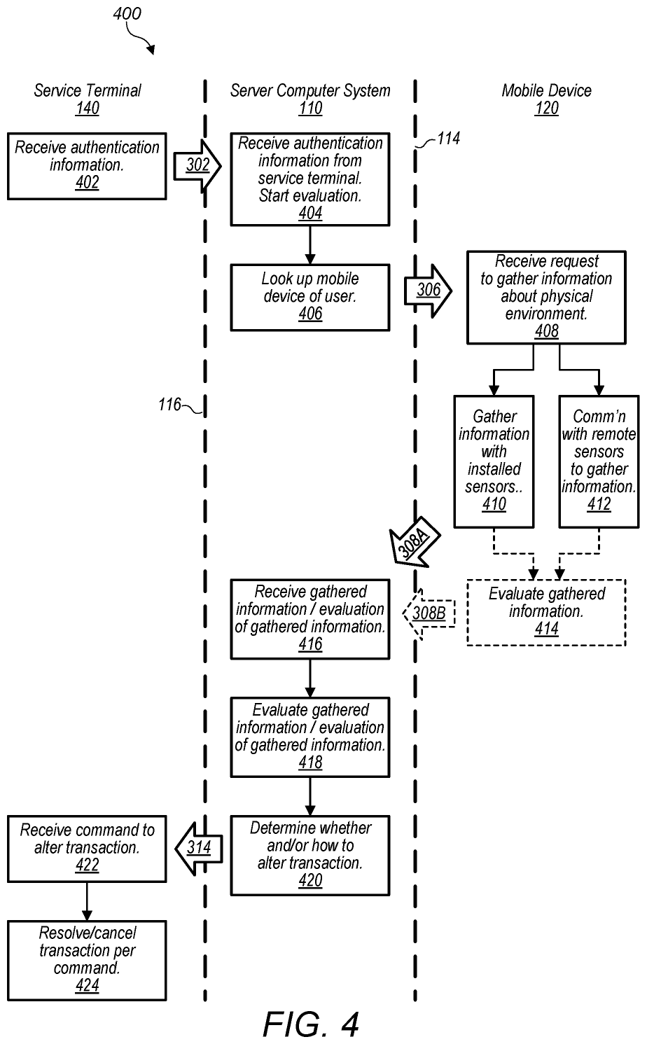

Referring now to FIG. 1, a block diagram of an exemplary embodiment of a computer system 100 is depicted. In various embodiments, computer system 100 includes a server computer system 110, a mobile device 120, and a service terminal 140. In various embodiments, a user 130 is associated with mobile device 120. In various embodiments, user 130 interacts with service terminal 140 to engage in a “transaction” with the service terminal 140 communicating with server computer system 110 during the transaction. As used herein, the term “transaction” refers to the interaction between user 130 from the moment the interaction begins (e.g., by user 130 inputting information to service terminal 140) until the moment the interaction ends (e.g., with a successful resolution of the transaction, with the transaction being cancelled by service terminal 140 as discussed herein). Moreover, as discussed in further detail with reference to FIG. 4, a transaction has one or more objects (e.g., access to a secure electronic resource) that are achieved at the resolution of the transaction (e.g., granting user 130 access to the secure electronic resource) or are not achieve if the transaction does not resolve successfully (e.g., service terminal 140 cancels the transaction).

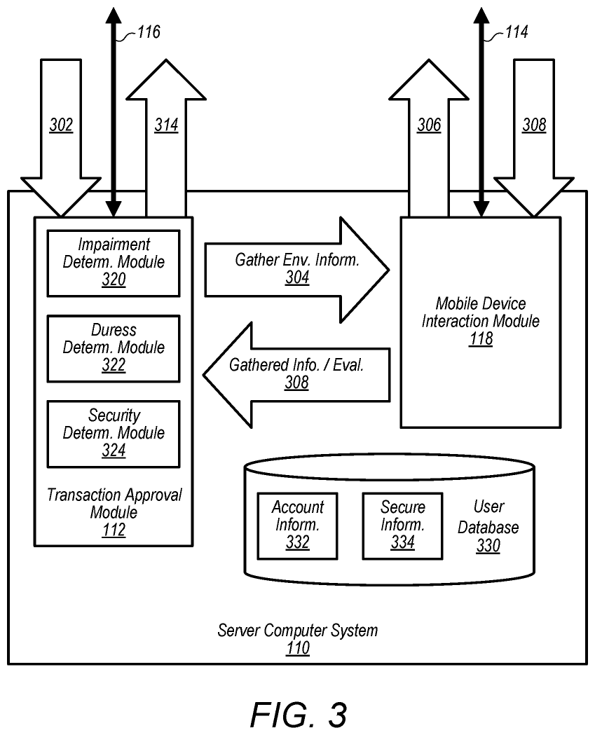

Server computer system 110 is one or more computer systems that communicate with mobile device 120 and service terminal 140 during the transaction as discussed herein. In various embodiments, server computer system 110 is remote from mobile device 120 and service terminal 140. Server computer system 110 may be implemented on a single computer system or a cloud of computer systems working in concert. As discussed in further detail in reference to FIG. 3, in various embodiments, server computer system 110 includes a transaction approval module 112 configured to perform the various actions associated with approving, rejecting, and/or altering the transaction discussed herein and a mobile device interaction module 118 configured to interact with mobile device 120. In various embodiments, server computer system 110 is configured to store account information for one or more users 130 that indicates, for ones of the users 130, that a given mobile device 120 is associated with a given user 130.

During a transaction initiated at service terminal 140, server computer system 110 is configured to receive authentication information related to a particular user 130 from service terminal 140. In various embodiments, prior to a resolution of the transaction and in response to the authentication information, server computer system 110 is configured to send a request to the particular mobile device 120 associated with user 130 to gather environmental information from one or more sensors 122, 124 in the physical environment 126 of mobile device 120. In various embodiments, still prior to the resolution of the transaction, server computer system 110 is configured to receive indications of gathered environmental information from mobile device 120, evaluate the gathered information, determine to alter the transaction based on the evaluating, and send a command altering the transaction to service terminal 140. In various embodiments, the received indications of gathered environmental information includes data from the various sensors 122, 124. Additionally or alternatively, the received indications of gathered environmental information includes evaluations of data from the various sensors 122, 124 (e.g., evaluations prepared by mobile device 120, evaluations prepared by other computing devices associated with remote sensors 124). Server computer system 110 is configured to communicate with mobile device 120 via communications link 114, which may be any of a number of wireless and/or wired communications mediums. Server computer system 110 is configured to communicate with service terminal 140 via communications link 116, which may be any of a number of wireless and/or wired communications mediums. In various embodiments, communication link 114 and/or communication link 116 comprise the Internet. Server computer system 110 is discussed in further detail in reference to FIG. 3 herein.

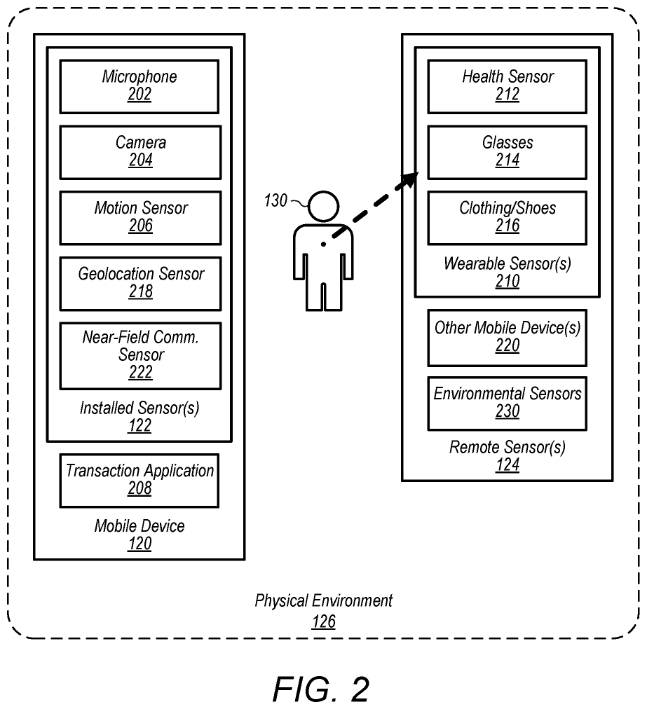

Mobile device 120 is any of a number of mobile computing devices including but not limited to a cellular phone, a smartphone, a tablet computer, or a laptop computer. In various embodiments, mobile device 120 is remote from server computer system 110. In various embodiments, mobile device 120 is remote from service terminal 140, but in other embodiments, service terminal 140 is within physical environment 126. In various embodiments, mobile device 120 includes one or more installed sensors 122. In various embodiments, mobile device 120 is configured to perform various tasks associated with a transaction associated with user 130 after the transaction has been initiated at service terminal 140 and prior to a resolution of the transaction. In such embodiments, mobile device 120 is configured to receive, from server computer system 110, a request for mobile device 120 to gather environmental information from one or more sensors 122, 124 in the physical environment 126 of mobile device 120. Mobile device 120 is configured to identify one or more sensors 122, 124 that are present in physical environment 126, send respective requests for environmental information to ones of the one or more sensors 122, 124 in physical environment 126, and receive environmental information from ones of the one or more sensors 122, 124. Mobile device 120 is further configured to send one or more indications of the environmental information to server computer system 110. Mobile device 120 is discussed in further detail herein in reference to FIG. 2.

Physical environment 126 of mobile device 120 is the area around mobile device 120. In various instances, various sensors 122, 124 and/or service terminal 140 are disposed within physical environment 126. In some embodiments, physical environment 126 is defined as a set radius around mobile device 120 (e.g., a 10-meter radius). In some embodiments, physical environment 126 is defined by the maximum range at which mobile device 120 is able to directly communicate with various sensors 122, 124 and/or service terminal 140.

As depicted in FIG. 1, one or more installed sensors 122 and one or more remote sensors 124 are disposed within physical environment 126. As further depicted in FIG. 1, service terminal 140 is also disposed within environment 126. It will be understood, however, that in various instances, no installed sensors 122 may be disposed within physical environment 126 (e.g., no installed sensors are present on mobile device 120), no remote sensors may be disposed within physical environment 126 (e.g., no remote sensors 124 are within the communications range of mobile device 120), and/or no service terminal 140 may be disposed within environment 126 (e.g., user 130 and mobile device 120 are more than 10 meters away from service terminal 140). Installed sensors 122 are one or more sensors installed on or within mobile device 120. Remote sensors 124 are one or more sensors that are physically separate from mobile device 120 and communicate with mobile device 120 using a wired and/or wireless communications media. Installed sensors 122 and remote sensors 124 are discussed in further detail herein in reference to FIG. 2.

User 130 is an individual who is attempting to perform, with service terminal 140, a transaction related to user 130. As depicted in FIG. 1 and FIG. 2, user 130 is present within physical environment 126 along with mobile device 120, service terminal 140, and one or more sensors 122, 124. In other instances, however, user 130 may be outside of the physical environment 126 (e.g., user 130 is not in possession of mobile device 120). In various instances, user 130 is the individual attempting to perform a transaction associated with user 130 with service terminal 140, but in other instances another person may be attempting to perform a transaction associated with user 130 (e.g., the other person is impersonating user 130, the other person has entered information into service terminal 140 related to user 130).

Service terminal 140 is one or more computer systems that are useable to initiate and resolve transactions with users 130. In various embodiments, service terminal 140 includes user interface 142 (e.g., a graphical user interface displayed on a touchscreen) configured to receive authentication information form user 130. The form and capabilities of service terminal 140 and user interface 142 vary, in various embodiments, according to the transaction(s) service terminal 140 is built to handle. In various embodiments where the transaction is a request to access a secure electronic resource (e.g., a secure website, a secure database within a network), service terminal 140 may be any of a number of computing devices (e.g., a laptop computer, desktop computer, tablet computer) useable to receive authentication information (e.g., usernames and passwords, biometric information) and to provide access to the secure electronic resources (e.g., by causing it to be shown on a display). Similarly, in various embodiments there the transaction is a request to access a secure physical area (e.g., a vault), service terminal 140 may be any of a number of devices (e.g., an RFID chip reader) useable to receive authentication information (e.g., a signal from an RFID identification card) and to provide access to the secure physical area (e.g., a turnstile). In various embodiments where the transaction is a financial transaction, service terminal 140 may be an automatic teller machine (e.g., for transactions relating to withdrawing or depositing cash or checking one or more bank account balances) or a computer system such as a laptop or tablet computer (e.g., for transactions relating moving money from one account to another or buying securities). As discussed herein and as depicted in FIG. 1, in various instances service terminal 140 is disposed within physical area 126, but in other instances service terminal 140 is outside physical area 126 (e.g., mobile device 120 is not within 10 meters of service terminal 140).

In various embodiments, user 130 initiates a transaction with service terminal 140 via user interface 142. In various embodiments, user 130 inputs authentication information to service terminal 140. For example, in various embodiments, user 130 inputs information via button presses or gestures on a touchscreen (e.g., by typing in a PIN on a keypad, by entering a username and password using a touchscreen), via a biometric interface (e.g., a thumbprint scanner), via a reader device (e.g., by using mobile device 120 to communicate with a near-field communication device coupled to service terminal 140, by swiping a card with a magnetic strip, by holding an RFID identification card up to an RFID reader), etc. As discussed herein, such entered authentication information is compared to stored or generated (e.g., a code generated using a seed value) authentication information that is associated with an account of user 130. If the authentication information does not match the reference authentication information, the transaction may be cancelled with service terminal 140 presenting a notification that the transaction has been cancelled.

In various embodiments, computer system 100 is operable to improve transactions between user 130 and service terminal 140. In various embodiments, such transactions are improved by determining whether user 130 is under duress or determining whether user 130 is intoxicated or otherwise impaired and altering the transaction accordingly (e.g., by canceling the transaction, by presenting incorrect information instead of the secure information at the resolution of the transaction). In various embodiments, such transactions are improved by providing additional security to the transactions (e.g., by ensuring that user 130 is alone, by requiring additional authentication before resolving the transaction, etc.). In various embodiments, during the transaction, server computer system 110 requests that mobile device 120 gather environmental information from one or more sensors (e.g., one or more installed sensors 122 and/or one or more remote sensors 124) in physical environment 126. As discussed herein, in various embodiments such environmental information includes information about user 130 (e.g., personal metrics) or information about physical environment 126 (e.g., whether additional people are near user 130). In various embodiments, mobile device 120 sends the gathered information to server computer system 110 for evaluation. In other embodiments, mobile device 120 evaluates the gathered information and sends an indication of the evaluation to server computer system 110. In embodiments, based on the evaluation server computer system 110 determines to alter the transaction and sends a command to service terminal 140 altering the transaction.

Referring now to FIG. 2, an expanded block diagram of the physical environment 126 of FIG. 1 is depicted in accordance with various embodiments. As depicted in FIG. 2, mobile device 120 (and its various components), user 130, and various remote sensors 124 are disposed within physical environment 126. It will be understood, however, that in various embodiments one or more of the components depicted in FIG. 2 are not present. For example, in some embodiments, mobile device 120 does not include motion sensor 206 and the only remote sensor 124 in physical environment is health sensor 212.

In various embodiments, mobile device 120 includes one or more installed sensors 122. Installed sensors 122 are one or more sensors installed on or within mobile device 120. In various embodiments, installed sensors 122 can include but are not limited to one or microphones 202, one or more cameras 204, one or more motion sensors 206, one or more geolocation sensors 218, one or more near-field communications sensors 222, or a combination. Using the one or more microphones 202, mobile device 120 is configured to capture audio information about physical environment 126 in various embodiments. Using the one or more cameras 204, mobile device 120 is configured to capture visual information about physical environment 126 in various embodiments. Using the one or more motion sensors 206, mobile device 120 is configured to capture information about how mobile device 120 is moving (or has moved) in various embodiments. Using the one or more geolocation sensors 218, mobile device 120 is configured to capture geolocation information for the mobile device 120 in various embodiments. Using the one or more near-field communications sensors 222, mobile device 120 is configured to communicate with near-field communications devices in physical area 126 (e.g., a near-field communication device coupled to service terminal 140) and to report a failed attempt to communicate with such near-field communications devices in various embodiments.

In various embodiments, after receiving a request to gather environmental information as discussed herein, mobile device 120 is configured to identify one of more of the installed sensors 122, request that the installed sensors 122 send captured environmental information (e.g., audio information, visual information, information about the movement of mobile device 120) for evaluation, receive such captured information from the installed sensors 122, and send the captured information and/or perform one or more evaluations based on the captured information as discussed herein and send indications of such evaluations to server computer system 110. In various embodiments, sending the environmental information includes sending information usable to determine to what kind of installed sensor(s) 122 gathered the environmental information and what the environmental information measures (e.g., a first indicator that a first set of environmental information is audio information captured by microphone 202, a second indicator that second set of environmental information is a visual information captured by camera 204).

In various embodiments, one or more remote sensors 124 are disposed within physical environment 126. These remote sensors 124 are one or more sensors that are physically separate from mobile device 120 and communicate with mobile device 120 using a wired and/or wireless communications media (e.g., Bluetooth, ZigBee, WiFi). In various embodiments, after receiving a request to gather environmental information as discussed herein, mobile device 120 is configured to identify one of more of the remote sensors 124 in physical environment 126. In some embodiments, ones of the remote sensors 124 include components that broadcast information about how to communicate with the remote sensor 124 (e.g., a unique address assigned to the remote sensor 124 such as a Media Access Control Address), and in such embodiments identifying these remote sensors 124 includes receiving such broadcast information. In other embodiments, identifying one of the remote sensors 124 includes mobile device 120 broadcasting a message commanding all remote sensors 124 in range to report in. Having identified one or more remote sensors 124, mobile device 120 is configured to send requests, to ones of the one or more remote sensors 124, for the one or more remote sensors 124 to send environmental information. Mobile device 120 is configured to receive such environmental information from the remote sensors 124, and send the environmental information and/or perform one or more evaluations based on the environmental information as discussed herein and send indications of such evaluations to server computer system 110. In various embodiments, sending the environmental information includes sending information usable to determine to what kind of remote sensor(s) 124 gathered the environmental information and what the environmental information measures (e.g., an first indicator that a first set of environmental information is heartrate information gathered by a health sensor 212, a second indicator that second set of environmental information is a weight measurement taken by a pressure sensor installed in the floor near service terminal 140).

In various embodiments, remote sensors 124 includes but is not limited to one or more wearable sensors 210, one or more other mobile devices 220, one or more environmental sensors 230, or a combination. In various embodiments, wearable sensors 210 are one or more sensors worn or carried by user 130. Such wearable sensors 210 include but are not limited to one or more health sensors 212, glasses 214, articles of clothing or shoes 216, or a combination. In various embodiments, the one or more health sensors 212 and/or articles of clothing or shoes 216 are configured to gather personal metrics about user 130 including but not limited to the heartrate, respiration rate, blood oxygen saturation, blood alcohol level, etc. of user 130. In various embodiments, health sensors 212 and/or articles of clothing or shoes 216 configured to gather information about the movements of user 130 (e.g., measuring the pace of the steps or other movement of user 130 which may be used to determine whether user 130 is staggering due to intoxication as discussed herein). Health sensors 212 may be worn in various places including but not limited to on wristbands, on chest straps, on the ends of fingers, etc. Articles of clothing or shoes 216 may be worn on any part of the body of user 130 and in various embodiments include sensors embedded in the articles of clothing or shoes 216 configured to capture the environmental information discussed herein. In various embodiments, glasses 214 are any kind of glasses (e.g., sunglasses, reading glasses, heads-up display glasses) worn on the face of user 130. In various embodiments, glasses 214 include one or more cameras useable to capture visual information about the eyes of user 130 and/or cameras useable to capture visual information about what user 130 is looking at.

In various instances, one or more other mobile devices 220 are disposed within physical environment 126. In various embodiments, mobile device 120 is configured to request that the other mobile devices 220 send environmental information to mobile device 120. In some of such embodiments, such environmental information includes information indicating a proximity of mobile device 120 to the one or more other mobile devices 220. In such embodiments, mobile device 120 is configured to send one or more indications of the proximity of mobile device 120 to the one or more other mobile devices 220.

In various instances, one or more environmental sensors 230 are disposed within physical environment 126. In various embodiments, environmental sensors 230 include but are not limited to cameras and/or microphones installed within physical environment 126 (e.g., a security camera installed near servicer terminal 140), a pressure sensor installed in the floor configured to measure the weight of user 130 and any other individuals also standing on the pressure sensor, or a combination. In such embodiments, the environmental sensors 230 are configured to gather environmental information about the environment around service terminal 140 and send such environmental information to mobile device 120 upon request.

In various embodiments, mobile device 120 includes a transaction application 208 configured to perform the various actions associated with facilitating the transaction discussed herein. In such embodiments, transaction application 208 may be installed on mobile device 120 by user 130 (or by another person). In embodiments, transaction application 208 is configured to configured to communicate with server computer system 110 and to cause mobile device to gather environmental information from sensors 122, 124 and send the gathered environmental information and/or perform one or more evaluations based on the environmental information as discussed herein and send indications of such evaluations to server computer system 110. In various embodiments, transaction application 208 uses cryptographic techniques (e.g., the use of encrypted messages, the use of certificates) to secure communication between mobile device 120 and server computer system 110.

In various embodiments, transaction application 208 may be configured to limit from which installed sensors 122 and/or remote sensors 124 that mobile device 120 requests environmental information. In various embodiments, such configurations are made by user 130 (e.g., user 130 has configured transaction application 208 request information from health sensor 212 but not to request audio information from microphone 202 installed on mobile device 120), maybe a manufacturer or network service provider for mobile device 120, and/or by a legal or regulatory authority (e.g., the U.S. Federal Communications Commission).

In various embodiments, mobile device 120 (e.g., using transaction application 208) is configured to evaluate the gathered environmental information and make one or more determinations about the transaction. For example, in some embodiments, mobile device 120 is configured to determine whether user 130 is under duress or intoxicated or the transaction presents a security risk based on the gathered environmental information. In various embodiments, mobile device 120 receives information about user 130 (e.g., personal metrics information indicating an elevated heart rate), about the presence of additional individuals in the physical environment 126 (e.g., information about the proximity of mobile device 120 to the one or more other mobile devices 220, audio information from microphone 202 indicative of more than one voice), about threatening events occurring within physical area 126 (e.g., aggressive voices or threatening words detected via microphone 202, a visual identification or a weapon by a camera 204) or a combination and determines, based on evaluating such information that user 130 is under duress. In such embodiments, mobile device 120 is configured to send one of more duress indications to server computer system 110. In other embodiments, mobile device 120 receives information about user 130 (e.g., visual or motion information indicative of staggered movements, audio information indicative of slurred speech, a measurement of blood alcohol level) and determines, based on evaluating such information that user 130 is intoxicated. In such embodiments, mobile device 120 is configured to send one of more intoxication indications to server computer system 110.

In various embodiments, the disclosed techniques enable mobile device 120 to receive and respond to a request to gather environmental information from server computer system 110 without server computer system 110 necessarily having visibility into what sensors 122, 124 are present within physical environment 126. For example, in various embodiments, server computer system 110 is configured to send a general request to mobile device 120 to gather environmental information, and mobile device 120 is configured to receive the request, identify one or more sensors 122, 124 that are responsive to the general request, gather environmental information from the identified sensors 122, 124, and report back to server computer system 110 about the gathered data by sending the gathered information and/or evaluations performed by mobile device 120 on the gathered information. Accordingly, in various embodiments, mobile device 120 is configured to improve transactions between user 130 and service terminal 140 without server computer system 110 knowing whether any sensors 122, 124 are present in physical environment 126 or what kind of sensors 122, 124 are present in physical environment 126 when the request to gather environmental information is sent to mobile device 120.

Referring now to FIG. 3, an expanded block diagram of the server computer system 110 of FIG. 1 is depicted in accordance with various embodiments. In various embodiments, server computer system 110 includes transaction approval module 112, mobile device interaction module 118, and user database 330. In such embodiments, transaction approval module 112 is configured to perform the various actions associated with approving, rejecting, and/or altering the transaction discussed herein and mobile device interaction module 118 is configured to interact with mobile device 120. In such embodiments, transaction approval module 112 communicates with service terminal 140 via communications link 116 and mobile device interaction module 118 communicates with mobile device 120 via communications link 114.

In various embodiments, transaction approval module 112 receives authentication information 302 from service terminal 140 via communications link 116. As discussed herein, service terminal 140 may be configured to receive various types of authentication information 302 from user 130 via any of number of input devices (e.g., a touchscreen, a keypad, a card reader, a biometric scanner) and send authentication information 302 to server computer system 110 via communications link 116. In various embodiments, authentication information 302 includes information indicative of one or more authentication factor such as knowledge factors (i.e., something the user knows such as a password), possession factors (i.e., something the user has physical access to such as a bank card), inherence factors (i.e., something that is part of or physically indicative of the user such as biometric indicators), or a combination.

In various embodiments, server computer system 110 includes one or more user databases 330. In such embodiments, user database 330 stores account information 332 for one of more users 130 used to facilitate the transaction. In various embodiments, account information 332 includes stored authentication information used to verify authentication information 302 (e.g., a stored hash of user 130's password for comparison to authentication information 302, previously stored biometric information for comparison to biometric information received as comparison to authentication information 302). Further, in embodiments, account information 332 for user 130 indicates a particular mobile device 120 that is associated with that particular user 130 (e.g., the phone number of mobile device 120). In some embodiments, user database 330 stores secure information 334 for user 130 that is associated with accounting formation 332 for one or more users 130. In various embodiments, access to secure information 334 is conditioned on a transaction resolving successfully. For example, secure information 334 may by an account balance of user 130's bank account, and access to this account balance is conditioned on the user 130 successfully using service terminal 140 to authenticate him or herself. In other embodiments, secure information 334 includes one or more databases of secured information (e.g., confidential information with restricted access).

After receiving authentication information 302, in various embodiments transaction approval module 112 verifies the authentication information 302 using account information 332. After successfully verifying authentication information 302 associated with a particular user 130, in various embodiments, transaction approval module calls mobile device interaction module 118 with command 304 to request that the particular mobile device 120 associated with the particular user gather environmental information as discussed herein. In various embodiments, mobile device interaction module 118 sends a request 306 to mobile device 120 via communications link 114 for mobile device 120 to gather environmental information from one or more sensors 122, 124 in the physical environment 126 of mobile device 120.

After mobile device 120 gathers such environmental information, in various embodiments, server computer system 110 receives gathered environmental information 308 from mobile device 120 via communications link 114. As discussed herein, in various embodiments, gathered environmental information 308 includes environmental information captured by sensors 122, 124 and gathered by mobile device 120 and/or evaluations on such environmental information. In various embodiments, gathered information 308 includes information usable to determine to what kind of sensor 122, 124 gathered the environmental information (e.g., a microphone 202, a health sensor 212) and what the environmental information measures (e.g., audio information, heart rate of user 130). In various embodiments, gathered information includes one or more measurements of user 130, one or more measurements of the physical environment 126 around the mobile device 120, or both.

As discussed herein in reference to FIG. 2, in some embodiments, gathered environmental information 308 includes personal metrics gathered about user 130 by one or more personal metrics sensors (e.g., a wearable sensor 210) worn by user 130. In some embodiments, gathered information 308 includes audio information, gathered by microphone 202 of the mobile device 120, visual information gathered by camera 204 of the mobile device 120, a geolocation of mobile device 120, one or more indications of failed near-field communication between mobile device 120 and service terminal 140, or a combination. In various embodiments, gathered environmental information 308 includes one or more user impairment indication based on evaluations performed by mobile device 120 of gathered environmental information, one or more duress indication based on evaluations performed by mobile device 120 of gathered environmental information, and/or one or more security risk indication based on evaluations performed by mobile device 120 of gathered environmental information.

In various embodiments, transaction approval module 112 receives gathered environmental information 308, performs one or more evaluations on it, and determines whether to alter the transaction based on the one or more evaluations. In various embodiments, such evaluations are performed using one or more modules 320, 322, 324 based on evaluating one or more measurements of the user, one or more measurements of the physical environment around the mobile device, or both. In various embodiments, impairment determination module 320 is configured to evaluate gathered information 308 to make a determination that user 130 is intoxicated based on the gathered environmental information 308 and to make a determination to alter the transaction is based on the determination that user 130 is intoxicated. For example, in various embodiments, gathered environmental information 308 includes but is not limited to information indicating that user 130 is walking erratically or stumbling (e.g., gathered by motion sensor 206, gathered by a wearable sensor 210), visual information indicating that user 130's eyes are dilated (e.g., gathered by glasses 214), audio information indicating that user 130's speech is incoherent or slurred (e.g., gathered by microphone 202, gathered by environmental sensor 230), an impairment indication from mobile device 120, or a combination. As discussed herein, in various embodiments, upon determining that user 130 is intoxicated, the transaction may be altered in number of ways including but not limited to canceling the transaction, reducing access to secured electronic resources (e.g., by limiting access to “top secret” designated information but still allowing access to a database generally), reducing a maximum amount of money that user 130 can transfer between bank accounts or withdraw from bank accounts, or a combination.

In various embodiments, duress determination module 322 is configured to evaluate gathered information 308 and to make a determination to alter the transaction is based on the determination that user 130 is under duress (e.g., being robbed by an assailant, being extorted). For example, in various embodiments, gathered environmental information 308 includes but is not limited to visual information indicating that another individual is standing in close proximity to user 130 and/or has a weapon (e.g., gathered by camera 204, glasses 214, and/or environmental sensors 230), audio information indicating that another individual is speaking to user 130 aggressively or has spoken words associated with a robber such as “give me all of the money in your account” (e.g., gathered by microphone 202, gathered by environmental sensor 230), information indicating that user 130 is not alone in front of service terminal 140 (e.g., gathered by environmental sensors 230, proximity information gathered from other mobile devices 220), a duress indication from mobile device 120, or a combination. As discussed herein, in various embodiments, upon determining that user 130 is under duress, the transaction may be altered in number of ways including but not limited to canceling the transaction, reducing access to secured electronic resources (e.g., by limiting access to “top secret” designated information but still allowing access to a database generally, by redirecting the transaction such that service terminal 140 grants access to an electronic sandbox instead of the secure electronic resource), reducing a maximum amount of money that user 130 can transfer between bank accounts or withdraw from bank accounts, present incorrect information instead of the secure information at the resolution of the transaction (e.g., by showing an incorrect bank account balance that is lower than the true bank account balance), by alerting law enforcement, or a combination.

In various embodiments, security determination module 324 is configured to evaluate gathered information 308 and to make a determination to alter the transaction is based on the determination that there is a security risk associated with the transaction. For example, in various embodiments, gathered environmental information 308 includes but is not limited to visual information indicating that another individual is standing in close proximity to user 130 (e.g., gathered by camera 204, glasses 214, and/or environmental sensors 230), audio information indicating that another individual is speaking to user 130 (e.g., gathered by microphone 202, gathered by environmental sensor 230), information indicating that user 130 is not alone in front of service terminal 140 (e.g., gathered by environmental sensors 230, proximity information gathered from other mobile devices 220), a security risk indication from mobile device 120, information indicating that the geolocation of mobile device 120 is not in proximity to service terminal 140 (e.g., gathered by geolocation sensor 218, determined as a result of one or more failed attempts to communicate with a near-field communication device coupled to service terminal 140), or a combination. In various instances, such information indicating that the geolocation of mobile device 120 is not in proximity to service terminal 140 potentially indicates that the individual attempting the transaction at service terminal 140 is not user 130 or that user 130 does not have his or her mobile device 120 in his or her possession. As discussed herein, in various embodiments, upon determining there is a security risk associated with the transaction, the transaction may be altered in number of ways including but not limited to canceling the transaction, reducing access to secured electronic resources (e.g., by limiting access to “top secret” designated information but still allowing access to a database generally, by redirecting the transaction such that service terminal 140 grants access to an electronic sandbox instead of the secure electronic resource), present incorrect information instead of the secure information at the resolution of the transaction (e.g., by dummy files), requesting additional authentication information before the resolution of the transaction (e.g., a second password, an additional form of identification), by alerting law enforcement, or a combination.

In various embodiments, after determining to alter the transaction, transaction approval module 112 sends command 314 altering the transaction to service terminal 140. As discussed herein, in various embodiments, the content of command 314 varies according to why kind of determination (e.g., duress determination, intoxication determination, security risk determination) has been made by server computer system 110 and/or mobile device 140. In various embodiments, command 314 can include instructions to service terminal 140 to cancel the transaction, reducing access to secured electronic resources (e.g., by limiting access to “top secret” designated information but still allowing access to a database generally, to redirect the transaction such that service terminal 140 grants access to an electronic sandbox instead of the secure electronic resource), to reduce a maximum amount of money that user 130 can transfer between bank accounts or withdraw from bank accounts, to present incorrect information instead of the secure information at the resolution of the transaction, to request additional authentication information before the resolution of the transaction, to alert law enforcement, or a combination. Alternatively, transaction approval module 112 is configured to send a command approving the transaction as-is (e.g., after computer system 100 has not made a duress, intoxication, or security risk determination).

In various embodiments, the disclosed techniques enable server computer system 110 to request that mobile device 120 gather environmental information that can be used to improve the transaction without server computer system 110 necessarily having visibility into what sensors 122, 124 are present within physical environment 126 when making the request. For example, in various embodiments, server computer system 110 is configured to send a general request to mobile device 120 to gather environmental information, and mobile device 120 is configured to receive the request, identify one or more sensors 122, 124 that are responsive to the general request, gather environmental information from the identified sensors 122, 124, and report back to server computer system 110 about the gathered data by sending the gathered information and/or evaluations performed by mobile device 120 on the gathered information. Accordingly, in various embodiments, mobile device 120 is configured to improve transactions between user 130 and service terminal 140 without server computer system 110 knowing whether any sensors 122, 124 are present in physical environment 126 or what kind of sensors 122, 124 are present in physical environment 126 when the request to gather environmental information is sent to mobile device 120.

Referring now to FIG. 4, a flowchart illustrates an embodiment of a transaction resolution process 400 between service terminal 140, server computer system 110, and mobile device 120 in accordance with various embodiments. At block 402, service terminal 140 receives authentication information (e.g., from user 130 via user interface 142). The authentication information 302 is sent to server computer system 110 by service terminal 140 via communications link 116. At block 402, server computer system 100 receives authentication information 302 relating to user 130 from service terminal 140 and starts a thread to evaluate physical environment 126. At block 406, server computer system 110 looks up information about user 130's mobile device 120 (e.g., from user database 330) and sends request 306 to mobile device 120 via communications link 114. At block 408, mobile device 120 receives request 306. In various instances and embodiments, mobile device 120 gathers environmental information from installed sensors 112 and/or communicates with remote sensors 124 to gather environmental information at blocks 410 and 412, respectively. In some embodiments, mobile device 120 sends gathered information 308 to server computing system 110 without performing any evaluations (indicated by arrow 308A in FIG. 4). In other embodiments, mobile device 120 evaluates the gathered environmental information at block 414 and sends gathered environmental information 308 and/or indications of evaluations performed on the gathered environmental information to server computer system 110 via communications link 114 (indicated by arrow 308B in FIG. 4). At block 416, server computer system 110 receives gathered environmental information 308 (which may include indications of evaluations performed on the gathered environmental information by mobile device 120 in various embodiments). At block 418, server computer system 110 evaluates gathered environmental information and at block 420, server computer system 110 determines (based on the evaluation) whether and/or how to alter the transaction. In instances where server computer system 110 determines to alter the transaction, server computer system 110 sends command 314 over communications link 116. At block 422, service terminal 140 receives command 314 to alter the transaction. At block 424, service terminal 140 resolves or cancels the transaction per command 314.

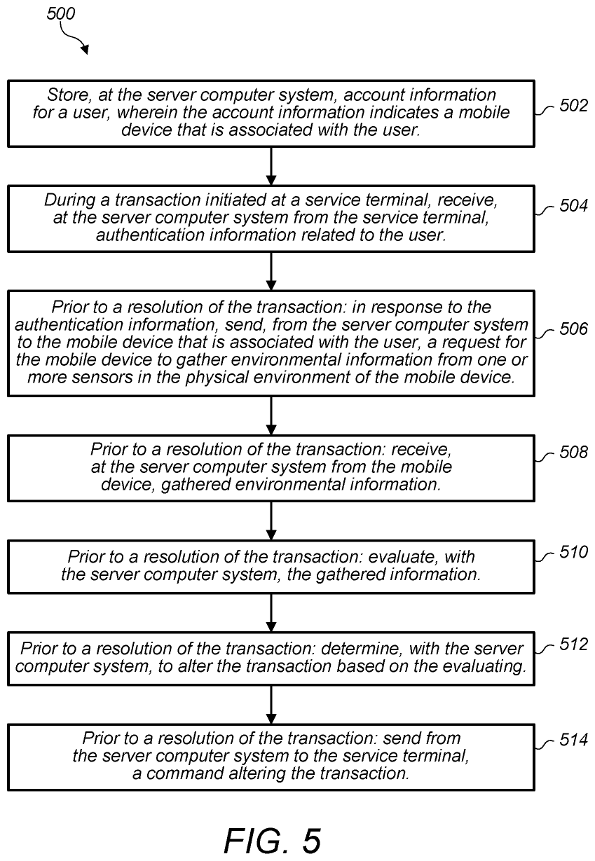

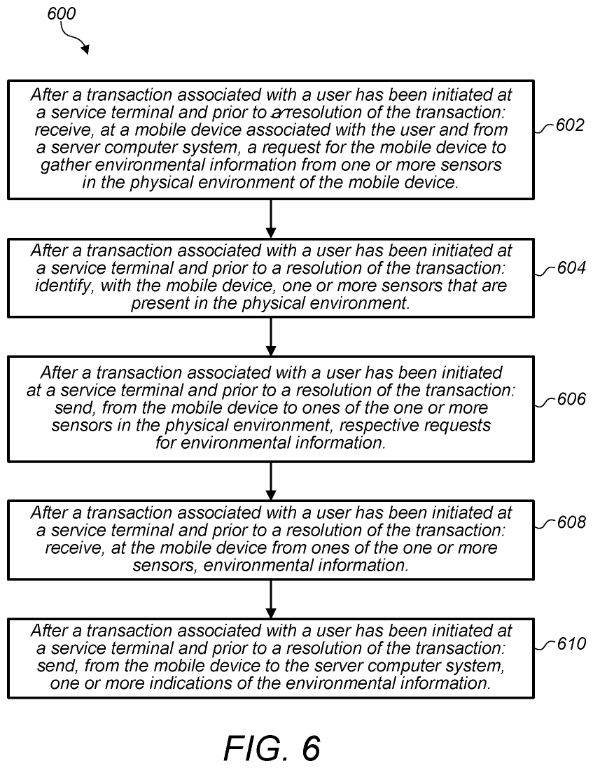

FIG. 5 and FIG. 6. illustrate various flowcharts representing various disclosed methods implemented with computer system 100. Referring now to FIG. 5, a flowchart illustrating an embodiment of a server portion 500 of a transaction resolution method is shown. In various embodiments, the various actions associated with the server portion 500 are performed with server computer system 110. At block 502, server computer system 110 stores account information 332 for user 130, wherein the account information 332 indicates a mobile device 120 that is associated with user 130. At block 504, during a transaction initiated at service terminal 140, server computer system 110 receives, from service terminal 140, authentication information 302 related to user 130. At block 506, prior to a resolution of the transaction and in response to authentication information 302, server computer system 110 sends, to mobile device 120 that is associated with user 130, a request 306 for mobile device 120 to gather environmental information from one or more sensors 122, 124 in physical environment 126 of mobile device 120. At block 508, prior to a resolution of the transaction, server computer system 110 receives, from mobile device 120, gathered environmental information 308. At block 510, prior to a resolution of the transaction, server computer system 110 receives, from mobile device 120, gathered environmental information 308. At block 510, prior to a resolution of the transaction server computer system 110 evaluates the gathered information 308. At block 512, prior to a resolution of the transaction server computer system 110 determines to alter the transaction based on the evaluating. At block 514, prior to a resolution of the transaction server computer system 110 sends, to service terminal 140, a command 314 altering the transaction.

Referring now to FIG. 6, a flowchart illustrating a mobile device portion 600 of a transaction resolution method is shown. In various embodiments, the various actions associated with the server portion 500 are performed with mobile device 120 after a transaction associated with user 130 has been initiated at service terminal 140 and prior to a resolution of the transaction. At block 602, mobile device 120 receives, from server computer system 110, a request 306 for mobile device 120 to gather environmental information from one or more sensors 122, 124 in the physical environment 126 of the mobile device 120. At block 604, mobile device 120 identifies one or more sensors 122, 124 that are present in physical environment 126. At block 606, mobile device 120 sends, to ones of the one or more sensors 122, 124 in physical environment 126, respective requests for environmental information. At block 608, mobile device 120 receives, from ones of the one or more sensors 122, 124, environmental information. At block 610, mobile device 120 sends to server computer system 110, one or more indications of the environmental information.

### Exemplary Computer System

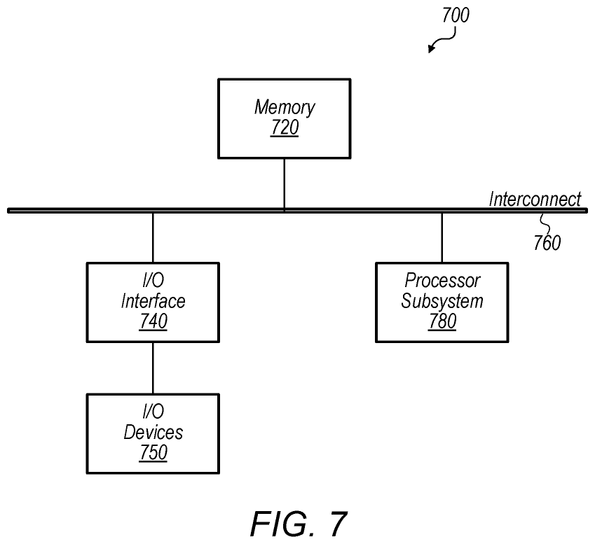

Turning now to FIG. 7, a block diagram of an exemplary computer system 700, which may implement the various components of computer system 100 (e.g., server computer system 110, mobile device 120, service terminal 140, remote sensors 124) is depicted. Computer system 700 includes a processor subsystem 760 that is coupled to a system memory 720 and I/O interfaces(s) 740 via an interconnect 760 (e.g., a system bus). I/O interface(s) 740 is coupled to one or more I/O devices 750. Computer system 700 may be any of various types of devices, including, but not limited to, a server system, personal computer system, desktop computer, laptop or notebook computer, mainframe computer system, tablet computer, handheld computer, workstation, network computer, a consumer device such as a mobile phone, music player, or personal data assistant (PDA). Although a single computer system 700 is shown in FIG. 7 for convenience, system 700 may also be implemented as two or more computer systems operating together.

Processor subsystem 760 may include one or more processors or processing units. In various embodiments of computer system 700, multiple instances of processor subsystem 760 may be coupled to interconnect 760. In various embodiments, processor subsystem 760 (or each processor unit within 760) may contain a cache or other form of on-board memory.

System memory 720 is usable to store program instructions executable by processor subsystem 760 to cause system 700 perform various operations described herein. System memory 720 may be implemented using different physical memory media, such as hard disk storage, floppy disk storage, removable disk storage, flash memory, random access memory (RAM—SRAM, EDO RAM, SDRAM, DDR SDRAM, RAMBUS RAM, etc.), read only memory (PROM, EEPROM, etc.), and so on. Memory in computer system 700 is not limited to primary storage such as memory 720. Rather, computer system 700 may also include other forms of storage such as cache memory in processor subsystem 760 and secondary storage on I/O Devices 750 (e.g., a hard drive, storage array, etc.). In some embodiments, these other forms of storage may also store program instructions executable by processor subsystem 760.

I/O interfaces 740 may be any of various types of interfaces configured to couple to and communicate with other devices, according to various embodiments. In one embodiment, I/O interface 740 is a bridge chip (e.g., Southbridge) from a front-side to one or more back-side buses. I/O interfaces 740 may be coupled to one or more I/O devices 750 via one or more corresponding buses or other interfaces. Examples of I/O devices 750 include storage devices (hard drive, optical drive, removable flash drive, storage array, SAN, or their associated controller), network interface devices (e.g., to a local or wide-area network), or other devices (e.g., graphics, user interface devices, etc.). In one embodiment, computer system 700 is coupled to a network via a network interface device 750 (e.g., configured to communicate over WiFi, Bluetooth, Ethernet, etc.).

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

What is claimed is:

1. A method, comprising:
storing, at a server computer system, account information for a user, wherein the account information indicates a mobile device that is associated with the user;
during a transaction initiated at a service terminal, receiving, at the server computer system from the service terminal, authentication information related to the user;
prior to a resolution of the transaction:
in response to the authentication information, sending, from the server computer system to the mobile device that is associated with the user, a request for the mobile device to gather environmental information from one or more sensors in a physical environment of the mobile device;
receiving, at the server computer system from the mobile device, gathered environmental information;
evaluating, with the server computer system, the gathered information;
determining, with the server computer system, to alter the transaction based on the evaluating; and
sending, from the server computer system to the service terminal, a command altering the transaction.

2. The method of claim 1,
wherein the account information is associated with secure information for the user;
wherein determining to alter the transaction includes determining to cause the service terminal to present incorrect information instead of the secure information at the resolution of the transaction; and
wherein the command altering the transaction is a command for the service terminal to present incorrect information instead of the secure information at the resolution of the transaction.

3. The method of claim 1,
wherein the transaction is a request to access a secure electronic resource;
wherein determining to alter the transaction includes determining to redirect the transaction such that the service terminal grants access to an electronic sandbox instead of the secure electronic resource; and
wherein the command altering the transaction is a command for the service terminal to resolve the transaction by granting access to the electronic sandbox instead of the secure electronic resource.

4. The method of claim 1,
wherein determining to alter the transaction includes determining to alter the transaction to add requesting additional authentication information before the resolution of the transaction; and
wherein the command altering the transaction is a command for the service terminal to request the additional authentication information before resolving the transaction.

5. The method of claim 1,
wherein determining to alter the transaction includes determining to cancel the transaction; and
wherein the command altering the transaction is a command for the service terminal to cancel the transaction.

6. The method of claim 1, wherein the gathered environmental information includes personal metrics gathered about the user by one or more personal metrics sensors worn by the user.

7. The method of claim 1, wherein the gathered information includes audio information, gathered by a microphone of the mobile device, visual information gathered by a camera of the mobile device, or both.

8. The method of claim 1,
wherein evaluating the gathered information includes making a determination that the user is under duress based on the gathered environmental information; and
wherein determining to alter the transaction is based on the determination that the user is under duress.

9. The method of claim 1,
wherein evaluating the gathered information includes making a determination that the user is intoxicated based on the gathered environmental information; and
wherein determining to alter the transaction is based on the determination that the user is intoxicated.

10. A non-transitory, computer-readable medium storing instructions that when executed by a server computer system cause the server computer system to perform operations comprising:
storing, at the server computer system, account information for a user, wherein the account information indicates a mobile device that is associated with the user;
during a transaction initiated at a service terminal, receiving, at the server computer system from the service terminal, authentication information related to the user;
prior to a resolution of the transaction:
in response to the authentication information, sending, from the server computer system to the mobile device that is associated with the user, a request for the mobile device to gather environmental information from one or more sensors in a physical environment of the mobile device;
receiving, at the server computer system from the mobile device, one or more indications of the gathered environmental information;
determining, with the server computer system, to alter the transaction based on the received indications; and
sending, from the server computer system to the service terminal, a command altering the transaction.

11. The computer-readable medium of claim 10,
wherein the one or more indications of the gathered environmental information includes one or more user impairment indications based on evaluations performed by the mobile device of the gathered environmental information; and
wherein determining to alter the transaction is based on the one or more impairment indications.

12. The computer-readable medium of claim 10, wherein determining, with the server computer system, to alter the transaction is based on evaluating one or more measurements of the user, one or more measurements of the physical environment around the mobile device, or both.

13. The computer-readable medium of claim 10,
wherein the one or more indications of the gathered environmental information includes a geolocation of the mobile device;
wherein determining, with the server computer system, to alter the transaction includes determining to cancel the transaction is based on the geolocation of the mobile device not matching a known location of the service terminal; and
wherein the command altering the transaction is a command to cancel the transaction.

14. The computer-readable medium of claim 10,
wherein the one or more indications of the gathered environmental information includes an indication of failed near-field communication between the mobile device and the service terminal;
wherein determining, with the server computer system, to alter the transaction includes determining to cancel the transaction is based on indication of failed near-field communication; and
wherein the command altering the transaction is a command to cancel the transaction.

15. A method comprising:
after a transaction associated with a user has been initiated at a service terminal and prior to a resolution of the transaction:
receiving, at a mobile device associated with the user and from a server computer system, a request for the mobile device to gather environmental information from one or more sensors in a physical environment of the mobile device;
identifying, with the mobile device, one or more sensors that are present in the physical environment;
sending, from the mobile device to ones of the one or more sensors in the physical environment, respective requests for environmental information;
receiving, at the mobile device from ones of the one or more sensors, environmental information; and
sending, from the mobile device to the server computer system, one or more indications of the environmental information.

16. The method of claim 15,
wherein the transaction is associated with a user;
wherein the one or more sensors in the physical environment include one or more personal metrics sensors worn by the user;
wherein receiving environmental information includes receiving personal metrics gathered about the user by the one or more personal metrics sensors worn by the user; and
wherein sending one or more indications of the environmental information includes sending an indication of the personal metrics.

17. The method of claim 15,
wherein the one or more sensors in the physical environment include a camera of the mobile device, a microphone of the mobile device, or both;
wherein receiving environmental information includes receiving visual information captured by the camera, audio information captured by the microphone, or both; and
wherein sending one or more indications of the environmental information includes sending an indication of the visual information, an indication of the audio information, or both.

18. The method of claim 15 further comprising:
after the transaction has been initiated at the service terminal and prior to the resolution of the transaction:
determining, with the mobile device, that the user is under duress or intoxicated based on the gathered information;
wherein the one or indications of environmental information includes an impairment indicator based on the determining.

19. The method of claim 15 further comprising:
wherein the one or more sensors in the physical environment include one or more additional mobile devices;
wherein receiving environmental information includes information indicating a proximity of the mobile device to the one or more additional mobile devices; and
wherein sending one or more indications of the environmental information includes sending an indication of the proximity of the mobile device to the one or more additional mobile devices.

20. The method of claim 15,
wherein the one or more sensors in the physical environment include a geolocation sensor usable to determine a geolocation of the mobile device;
wherein receiving environmental information includes receiving an indication of the geolocation of the mobile device; and
wherein sending one or more indications of the environmental information includes sending the indication of the geolocation of the mobile device.
